(19) United States
(12) Patent Application Publication
(10) Pub. No.: US 2024/0055132 A1
(43) Pub. Date: Feb. 15, 2024

(54) SYSTEMS AND METHODS TO ASSESS HEALTH INFORMATION FOR A SUBJECT BASED ON SUBJECT-INITIATED SESSIONS WITH ONLINE APPLICATIONS

(71) Applicant: Solsten, Inc., St. Paul, MN (US)

(72) Inventor: Joseph Jack Schaeppi, Maple Grove, MN (US)

(21) Appl. No.: 17/888,218

(22) Filed: Aug. 15, 2022

(51) Int. Cl. G16H 50/30; G16H 10/20; G16H 10/60

(52) U.S. Cl. CPC G16H 50/30; G16H 10/20; G16H 10/60

(57) ABSTRACT

Systems and methods to assess health information for a subject based on subject-initiated sessions with online applications are disclosed. Exemplary implementations may: store, in electronic storage, psychological profiles of subjects, application usage information for the subjects, and/or stated information for the subjects; receive, from an online application, session usage information for a subject responsive to the subject initiating a session with the online platform; and determine, in an ongoing manner during the session, health assessment information for the subject based on the session usage information, the application usage information, a psychological profile of the subject, and/or the stated information.

### FIELD OF THE DISCLOSURE

The present disclosure relates to systems and methods to assess health information for a subject based on subject-initiated sessions with online applications.

### BACKGROUND

Healthcare providers and/or caregivers may seek to provide subjects with most appropriate healthcare to address their conditions and/or needs. However, current technologies and/or methods may not accurately assess the subjects' current mental health and/or emotional health at a particular point in time. The current technologies and/or methods may include questionnaires, disclosures, logs kept by the subject, and/or other information reliant on communication from the subject and/or their caregivers.

### SUMMARY

The present disclosure may provide accurate or more accurate determination of health assessment information for a given subject by analyzing how and when the given subject previously interacts with content provided by online applications in addition to what particular content, who they interact with, and where they initiate and interact with the online applications. Additionally, the health assessment information may be determination by analyzing a current session during which the given subject interacts with one or more of the online applications. During such interactions and the session, the given subject may make decisions and/or selections subconsciously as opposed to deliberately attempting to convey a less than truthful current mental health and/or emotional health. Thus, healthcare may be most appropriately determined for the given subject given their more accurate health assessment information.

One aspect of the present disclosure relates to a system configured to assess health information for a subject based on subject-initiated sessions with online applications. The system may include one or more hardware processors configured by machine-readable instructions. Server(s) may be configured by machine-readable instructions. The machine-readable instructions may include one or more instruction components. The instruction components may include computer program components. The instruction components may include one or more of session usage information receiving component, health assessment information determination component, and/or other instruction components.

Electronic storage may be configured to store psychological profiles of subjects, application usage information for the subjects, interaction information, stated information for the subjects, and/or other information. The psychological profiles may be determined based on the application usage information, the interaction information, the stated information previously obtained for the subjects, and/or other information.

The session usage information receiving component may be configured to receive, from an online application, session usage information and/or other information for a subject responsive to the subject initiating a session with the online application. The session usage information may characterize current use of the online application by the subject.

The health assessment information determination component may be configured to determine, in an ongoing manner during the session, health assessment information for the subject based on the session usage information, the application usage information, a psychological profile of the subject, the stated information, and/or other information.

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured to assess health information for a subject based on subject-initiated sessions with online applications, in accordance with one or more implementations.

FIG. 2 illustrates a method to assess health information for a subject based on subject-initiated sessions with online applications, in accordance with one or more implementations.

FIG. 3 illustrates an example implementation of the system configured to assess health information for a subject based on subject-initiated sessions with online applications, in accordance with one or more implementations.

### DETAILED DESCRIPTION

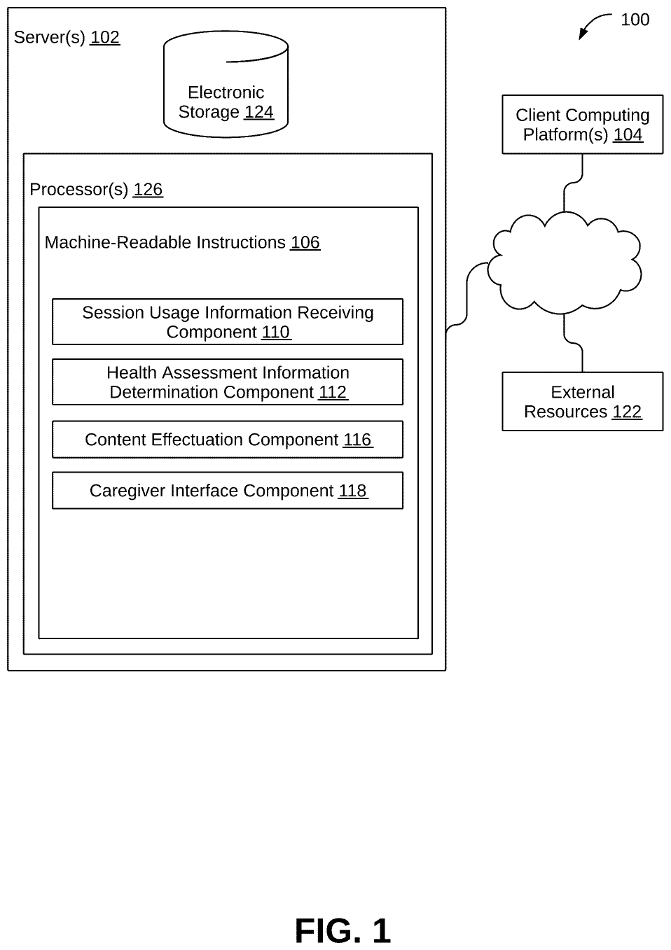

FIG. 1 illustrates a system 100 configured to assess health information for a subject based on subject-initiated sessions with online applications, in accordance with one or more implementations. In some implementations, system 100 may include one or more servers 102. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via client computing platform(s) 104.

Server(s) 102 may be configured by machine-readable instructions 106. Machine-readable instructions 106 may include one or more instruction components. The instruction components may include computer program components. The instruction components may include one or more of session usage information receiving component 110, health assessment information determination component 112, content effectuation component 116, caregiver interface component 118, and/or other instruction components.

Electronic storage 124 may be configured to store psychological profiles of subjects, application usage information for the subjects, interaction information related to the application usage information, stated information for the subjects, and/or other information. The subjects may be patients or people who require assistance. The psychological profiles may characterize the subjects psychologically. The application usage information may characterize how and when the subjects use individual applications on client computing platforms 104 and/or within online platforms. The interaction information related to the application usage information may characterize content engaged with or interacted with by the subjects, how the subjects engage with the content, and/or other interaction information.

Individual online platforms may provide access to a plurality of applications, online and offline. Client computing platforms 104 (e.g., smartphone, laptop, game console) may provide access to a plurality of the applications. In some implementations, the application provided by client computing platform 104 may be initially provided by online platforms (e.g., downloaded from the online platforms). In some implementations, an online platform and a digital environment provided thereof may be referred to as the application. The term “online application” used herein may refer to any application, online and offline, and may be used interchangeably with the term “application”.

The digital environment and/or individual applications may provide simulated spaces or views of a virtual space. Individual simulated spaces may have a topography, express ongoing real-time interaction by one or more subjects, and/or include one or more objects positioned within the topography that are capable of locomotion within the topography. In some instances, the topography may be a 2-dimensional topography. In other instances, the topography may be a 3-dimensional topography. The topography may include dimensions of the space, and/or surface features of a surface or objects that are “native” to the space. In some instances, the topography may describe a surface (e.g., a ground surface) that runs through at least a substantial section of the space. In some instances, the topography may describe a volume with one or more bodies positioned therein (e.g., a simulation of gravity-deprived space with one or more celestial bodies positioned therein). The instance executed by the computer components may be synchronous, asynchronous, and/or semi-synchronous.

By way of non-limiting example, the content may include one or more of a character, a game, a game asset, a recommendation, a suggestion, a promotion, and/or other content. The character may refer to an object (or group of objects) present in a virtual space that corresponds to an individual subject (e.g., an avatar) and/or are controlled by the subject. In some implementations, the character may not correspond to an individual subject but rather provide information (e.g., the recommendation, the suggestion) to the subject. The game asset may include a virtual item, a virtual resource (e.g., weapon, tool), of in-game powers, in-game skills, in-game technologies, and/or other game assets. The recommendation may include a particular selection and/or action that the subject is advised to select and/or do. The suggestion may include particular ideas, plans, and/or strategies for the subject to consider executing, following, and/or is determined they will enjoy. The promotion may include discount codes, coupons, bonuses, and/or other promotions of the virtual items, products, and/or services that the subject may utilize. By way of non-limiting example, products and/or services may relate to beauty (e.g., skincare, makeup), home improvement, decoration, clothing, accessories, technology, kitchen, and/or other categories.

The above description of the manner in which views of the virtual space are provided is not intended to be limiting. The virtual space may be expressed in a more limited, or richer, manner. For example, views determined for the virtual space may be selected from a limited set of graphics depicting an event in a given place within the virtual space. The views may include additional content (e.g., text, audio, pre-stored video content, and/or other content) that describes particulars of the current state of the place, beyond the relatively generic graphics. For example, a view may include a generic battle graphic with a textual description of the opponents to be confronted. Other expressions of individual places within the virtual space are contemplated.

The psychological profiles may be determined based on or otherwise derived from the application usage information, the interaction information, the stated information previously obtained for the subjects, and/or other information. The psychological profiles may include sets of psychological parameter values to the psychological parameters for the individual subjects. By way of non-limiting example, the psychological parameter values of the psychological parameters may be a number score on a predetermined range unique to each psychological parameter, a letter score, and/or other type of value than may characterize a particular subject as whole.

Parameters, such as psychological parameters are described herein, may specify measurable, recordable, and/or determined information. The parameter values corresponding to the parameters may be a particular value, numerical or non-numerical, that characterizes the content, the users, or respective element that the parameter value is described in relation to.

The psychological parameter values may characterize, by way of non-limiting example, achievement motivation, motivation, personality inventory, cultural values, competitiveness, positive and negative affect before, during, and/or after engagement with the digital experience (i.e., emotions), communication style, personal values, daily routines/activities, life/gaming pain points, life/gaming hopes and aspirations, wellbeing, user experience, gaming/experience using time, subscription behavior, affinity information, personality, emotional style, goal orientation, goal commitment, ego and task orientation, relatedness, sense of community, social influence, social identity, group identification, we-identity, quality of life, satisfaction with life, work-related quality of life, mindfulness, happiness, emotional intelligence, self-awareness/internal awareness, external awareness, connectedness to nature, social connectedness, social bonding, perceived stress, depression, anxiety, decision-making style, thinking style, critical thinking, cognitive approach to learning, learning style, attributional style, internality-externality, stability-instability, global-specific, creativity, curiosity, playfulness, exploration, mental strength, grit, flourishing, gratitude, inspiration, spirituality, hedonism, materialism/material values, perceptions, sentiments, and/or other psychological parameters.

Achievement motivation may include compensatory effort, competitiveness, confidence in success, dominance, eagerness to learn, engagement, fearlessness, flexibility, flow, goal setting, independence, internality, persistence, preference in difficult tasks, pride in productivity, self-control, status orientation, ambition, self-assurance, and/or other psychological parameters. Motivation may include mastery, purpose, autonomy, and/or other psychological parameters.

Personality inventory may include neuroticism, openness, conscientiousness, extraversion, and agreeableness and/or other psychological parameters. Neuroticism may include anxiety, impulsiveness, vulnerability, and/or other psychological parameters. Openness may include fantasy, feelings/empathy, action, and/or other psychological parameters. Conscientiousness may include achievement striving, competence, self-discipline, and/or other psychological parameters. Extraversion may include warmth assertiveness, activity, and/or other psychological parameters. Agreeableness may include trust, altruism, modesty, and/or other psychological parameters.

Cultural values may include individualism, indulgence, long term orientation, masculinity, power distance, uncertainty avoidance, and/or other psychological parameters. Competitiveness may include avoidant, collaborative, competitive affectivity, dependent, dominant, general competitiveness, independent, personal enhancement, and/or other psychological parameters.

Positive and negative affect before, during, and/or after engaging in the digital experience may include hostility, joviality, negative emotions, positive emotions, sadness, self-assurance, and/or other psychological parameters. Communication style may include feeler, intuitor, sensor, thinker, and/or other psychological parameters.

Wellbeing may include social wellbeing, psychological wellbeing, physical wellbeing, physical activity, sleep, bounded reciprocity, resilience grit, and/or other psychological parameters.

Personality may include anger, hostility, depression, self-conscientiousness, excitement-seeking, positive emotions, gregariousness, ideas, values, aesthetics, tender-mindedness, straightforwardness, compliance, deliberation, order, dutifulness, and/or other psychological parameters.

Emotional style may include resilience, outlook, social intuition, self-awareness, sensitivity to context, attention, and/or other psychological parameters.

Goal orientation may include mastery approach/learning goal orientation, performance approach/performance goal orientation, performance avoid/avoidance goal orientation, and/or other psychological parameters.

Work-related quality of life may include structure, boundaries, focus, efficiency, information provision, communication, psychological support, stress at/from work, psychological safety, connectedness with team, motivation to work, adaptability, job/career satisfaction, control at work, home-work interface, general wellbeing, working conditions, and/or other psychological parameters.

Mindfulness may include observing, describing, acting with awareness, non-judgment, non-reactivity, and/or other psychological parameters.

Emotional intelligence may include emotion perception, emotion expression, emotion management, emotion regulation, impulse control, relationships, stress management, and/or other psychological parameters.

Social connectedness may include social connectedness, loneliness, membership self-esteem, private self-esteem, public self-esteem, identity self-esteem, interdependent self, independent self, social avoidance, social distress, and/or other psychological parameters.

Decision-making style may include respected, confident, spontaneous, dependent, vigilant, avoidant, brooding, intuitive, anxious, and/or other psychological parameters.

Thinking style may include intuitive, experiential, analytical, rational, and/or other psychological parameters.

Cognitive approaches to learning may include avoidant, participative, competitive, collaborative, dependent, independent, and/or other psychological parameters.

Learning style may include visual (spatial), aural (auditory-musical), verbal (linguistic), physical (kinesthetic), logical (mathematical), social (interpersonal), solitary (intrapersonal), and/or other psychological parameters.

Mental strength may include tenacity, confidence, optimism, adaptability, self-awareness, reliability, responsibility, well-being, and/or other psychological parameters.

Flourishing may include positive emotion, engagement, relationships, meaning, accomplishment, health, loneliness, and/or other psychological parameters.

The stated information may include answers of the users to questions presented to the users, descriptions provided by the users that describe their psychological traits and/or themselves generally, one or more pieces of content (e.g., music genre, color palette, television shows, etc.) the users like or have an affinity to, one or more pieces of content the users dislike or have an aversion to, and/or other stated information that the users may provide regarding themselves. The questions may be related to psychological attributes, real-world interactions, real-world likes and dislikes, and/or other questions.

The application usage information may include values to screen time on the individual applications, battery usage of the individual applications, Internet usage of the individual applications, location usage by the individual applications, times of installations of the individual applications, the application types of installations of the individual applications, costs of the installations, times of initiations of the individual applications, times of terminations of the individual applications, amount of notifications from the individual applications, notification types of the notifications, response to the notifications, cross-application information usage, times of in-application purchases and in-application sales, item type of the in-application purchases, the item type of the in-application sales, the application types of the applications initiated, and/or other usage parameters.

The screen time and/or battery usage may indicate an amount of time that the subject spends on client computing platform 104 collectively over a period of time and/or an amount of time the subject spends on individual applications. The Internet usage may indicate an amount of time and when the subject is online (i.e., using the Internet) and offline within the applications. The location usage may indicate how often a location of client computing platform 104 is requested of the subject and/or authorized for use by the subject. The location usage may indicate where the subject is during use of the online applications and/or movement during the use of the online applications (e.g., travelling by car, static at work). The response to the notifications and the notification types thereof may indicate what types of notifications the subject often responds to and ignores. The notification types may include, by way of non-limiting example, a time sensitive notification (e.g., a sale countdown for a virtual or physical item), a question notification, a feedback request (e.g., experience survey), a contest or sweepstakes entry, an incentive (e.g., if you provide feedback, you get a gift card), a reminder, informational (e.g., weather alert), a promotion/deal, and/or other notification types.

The interaction information may include the content engaged with by the subjects, how the individual subjects engaged with the content, and/or other interaction information. The content engaged with by the individual subjects may be related to the online platforms or the individual online applications that provide the content. That is, for example, the content provided by an online gaming platform may relate to online games (e.g., virtual goods, virtual mini games, etc.). In some implementations, the content engaged with by the individual subjects may not be related to the online platforms that provide the content. Meaning, the content may direct the subject to a different online application or online platform.

The engagement by the individual subjects may define behavior patterns of the individual subjects with or based on the content. The behavior patterns of the individual subjects may include individual actions, sets of actions, ordered sets of actions, time spent by the individual subjects engaging with the content and/or the online platforms, spending patterns of the subjects, completed tasks by the individual subjects, uncompletion tasks by the individual subjects, failure of tasks by the individual subjects, and/or other behavior patterns. In some implementations, the behavior patterns may include multiple of the individual actions, the sets of actions, and the ordered set of actions. The actions may include one or more of a purchase based on the content, a sale, a trade, a donation, a subject selection of the content, gameplay (e.g., mini-game, battle, competition, etc.) based on the content, communication of the individual subjects with other particular subjects or users, frequent interaction with the content, formation of alliances by the subjects, and/or other actions. The spending patterns may indicate an amount of currency (e.g., real-world money, virtual money, points, etc.) spend, an amount of currency earned, an amount of currency donated, and/or other indications.

In some implementations, the interactions of the subjects with the digital environments and/or the behavior patterns of the subjects may be correlated with psychological attributes, i.e., the psychological parameter values to the psychological parameters, as described in U.S. application Ser. No. 16/894,522 entitled “SYSTEMS AND METHODS TO CORRELATE USER BEHAVIOR PATTERNS WITHIN AN ONLINE GAME WITH PSYCHOLOGICAL ATTRIBUTES OF USERS” Attorney Docket No. 01TT-064002, the disclosure of which is incorporated by reference in its entirety herein.

Session usage information receiving component 110 may be configured to receive session usage information for a subject responsive to the subject initiating a session with an online application. In some implementations, the online platforms may provide access or host the online applications for the subjects via client computing platform 104 (e.g., suite of applications, mobile applications, desktop applications). In some implementations, the individual online applications may be provided by the online platforms and accessible by the subjects via web browsers (i.e., web version of a desktop game, online games via websites).

In some implementations, the session may be initiated within a single online application. In some implementations, the session may be initiated external to the single online application. For example, a particular application on client computing platform 104 of the subject or a particular website may be utilized by the subject to initiate the session. Subsequently, the session may include session usage information from one or more online applications that the subject chooses.

The online applications, and application types thereof, may include game applications, educational applications, reading applications, music applications, social networking applications, entertainment applications, fitness applications, business applications, shopping applications, food & drink applications, among other applications.

The session usage information may be received from individual online applications. The session usage information may characterize current use of the online application by the subject. The current use of individual online applications may refer to how the subject is interacting with the online application and content thereof, when the subject is interacting or using the online application, what content the subject is interacting with, what other subjects or people they subject is communicating or interacting with via the online application, and/or other uses during a particular time range. The time range may be from a first date to a second date, a particular time span of a day (e.g., 6 PM-10 PM), the particular time span of a day over multiple days, the particular time span of a day on particular days of the week, the particular time span of a day on specific dates, and/or other time ranges.

The current use may be a particular time period during which the subject is being evaluated, monitored, measured, or otherwise examined via their use of the online applications. Such session usage information may be utilized by medical professionals (e.g., physicians, psychologists, psychiatrists), caregivers, and/or others to provide healthcare.

The session usage information may include game mechanics within the online application the subject employed, the game mechanics within the online application the subject avoided, one or more games chosen to be played, one or more of the games disregarded, session results, read literature, unread literature, social interactions, and/or other session usage information.

The game mechanics may include alternating turns in the one or more games, action points, playing cards, capturing, catch-up progression, dice, movements, resource management, risk and reward, role-playing, game modes (e.g., single-player, multiplayer), and/or other novel or known game mechanics. In some implementations, different ones of the games and/or the content provided by the online application may employ different game mechanics. Thus, the subject may initiate and utilize different game mechanics within the online application. Conversely, the subject may disregard some of the game mechanics by disregarding some of the games and/or the content within the online application.

The session results may include wins, losses, virtual items acquired, virtual items dropped, level attempts, levels achieved, characters played as, characters battled and subjects thereof, in-application or in-game movements, and/or other session results. The wins and losses may be of the one or more games, and/or other wins and losses. The virtual items acquired and dropped may be from the one or more games won and lost, respectively. For example, the virtual items may include tokens, stars, digital currency, virtual goods (e.g., weapons, tools, food, clothing, etc.), and/or other virtual items. The in-application or in-game movements may include tracked movements around a map of a game, movements along a game board, movements of playing cards (e.g., Solitaire), action or battle movements (e.g., jump-shoot, flip and kick, sword combinations, punch combinations), and/or other movements. The games within the online applications may be mini-games within an encompassing game provided by the online application, distinct mini-games, and/or other games. The games may have a game type. For example, the game types may include a puzzle game, a matching game, a racing game, a strategy game, a role-playing game, a party game, a multiplayer battle game, an action-adventure game, a survival and horror game, a sports simulation game, a shooter game, and/or other game types.

The literature may include manuals, books, articles, policies, messages, law, and/or other literature. The social interactions may include direct messages between the subject and other users or subjects, audio communication between one or more other users and/or subjects, video communications between the subject and one or more other users and/or subjects, public messages between the subject and one or more other users and/or subjects (e.g., comments under a post), selecting a virtual button that indicates a reaction (e.g., like, dislike, agreement, disagreement, laughter), and/or other social interactions.

In some implementations, the session usage information received may be stored in electronic storage 124 in associated with the subject. In some implementations, electronic storage 124 may be organized to store subject summaries for the subjects. An individual subject summary may include identifying information, the psychological profile of a given subject, the application usage information, the interaction information, past session usage information, the healthcare, and/or other information determined about or for the given subject.

In some implementations, indication receiving component 114 may be configured to receive an indication that the session has been initiated by the subject. The indication may be presented via a caregiver interface of a client computing platform 104 associated with a caregiver and/or via an interface of client computing platform 104 that the subject initiated the session on. In some implementations, the indication may include identifying information for the subject. Thus, subsequent to and based on the indication, the session usage information may be received and stored to electronic storage 124 in association with the subject. The identifying information may include a name, a date of birth, a medical identifier, other unique identifiers (e.g., a particular medical office record number), social security information, address, phone number, email, and/or other information that may facilitate confirmation of the subject's identity.

In some implementations, content effectuation component 116 may be configured to effectuate, via the online application, content that require one or more responses from the subject. By way of non-limiting example, the content may include timed challenges, written response questions, riddles, visual games, auditory games, memory games, and/or other content requiring responses. In some implementations, the content may be generated and/or selected by one or more of the caregivers of the subject. In some implementations, the content may be an item within the online application. The responses may be included in the stated information upon receipt. Thus, determining health assessment information may be further based on the one or more responses that are provided during the session.

Health assessment information determination component 112 may be configured to determine the health assessment information for the subject based on the session usage information, the application usage information, the interaction information, the psychological profile of the subject, the stated information, and/or other information related to the subject. The health assessment information may be determined in an ongoing manner during the session. The term “ongoing manner” as used herein may refer to continuing to perform an action (e.g., determine) periodically (e.g., every 30 seconds, every minute, every hour, etc.) until receipt of an indication to terminate. The indication to terminate may include powering off client computing platform 104, a time of day during which the health assessment information is not determined, and/or other indications of termination. In some implementations, the health assessment information may be determined upon completion or termination of the session. The session may be a defined amount of time, a minimum amount of time, and/or a maximum amount of time. In some implementations, the subject must complete the minimum amount of time. In some implementations, the subject may not continue the session over the maximum amount of time. In some implementations, the defined amount of time may be defined by the caregiver(s) via caregiver interface(s).

In some implementations, the health assessment information may include a current mental state, a current emotional state, changes for the psychological parameter values to the psychological parameters, that comprise the psychological profile and/or other health assessment information. The health assessment information may indicate new disorders, illnesses, and/or conditions of the subject. In some implementations, the health assessment information may indicate changes in identified disorders, illnesses, and/or conditions of the subject. In some implementations, the health assessment information may indicate probable or impeding disorders, illnesses, and/or conditions of the subject. By way of non-limiting example, the disorders, illnesses, and/or conditions may include insomnia, eating disorders, addictions, mood disorders, learning disabilities (e.g., dyslexia, dyscalculia, dysgraphia, etc.), personality disorders, psychotic disorders, and/or other disorders, illnesses, and/or conditions.

The current mental state may be how the subject processes and/or understands their thoughts and actions. The current mental state may include one or more of psychological well-being, social well-being, and/or emotional well-being, and/or other well-being of the subject, collectively. The current mental state may be reflected or represented in one or more of the subject's sleeping patterns (e.g., not enough sleep, excessive sleeping, 8-hour sleep, restlessness), eating patterns (e.g., overeating, under-eating, purging), energy, mood, substance consumption, and/or other representations.

Some or all of the psychological parameter values to the psychological parameters may define, closely define, or facilitate determining the psychological well-being, the social well-being, and the emotional well-being, collectively, of the subject. In some implementations, sets of the psychological parameters and their psychological parameter values for the subject and particular interactions with the content and/or specific content during the session may characterize the representations of the current mental state and thus the psychological well-being, the social well-being, and the emotional well-being of the subject. For example, a first set of psychological parameters and the psychological parameter values thereof, and first content interacted with during the session may characterize the sleeping pattern of the subject and indicate that the subject has insomnia. Additionally, the application usage information, such as the screen time, may additionally indicate that the subject has insomnia, cognition of the subject, mental health characteristics, and/or other disorders, illnesses, and/or conditions. By way of non-limiting example, the mental health characteristics may include inability to deal with stress, violence, hostility, anger, trouble relating to others or situations, low energy, inability to concentrate, and/or other mental health characteristics.

The current emotional state may be having awareness of emotions and having the ability to manage and express the emotions appropriately, or lack thereof. The current emotional state may be reflected or represented in one or more of the subject's relationships with people (e.g., friends, family, authority, strangers), communication with the people, reactions to receiving communications from the people, reactions to events or circumstances (e.g., failing a test, accomplishing a goal), and/or other representations. In some implementations, some of the psychological parameter values to one or more of the psychological parameters related to the emotional intelligence, the emotional style, the flourishing psychological parameters, and/or other psychological parameters may particularly facilitate determination of the current emotional state in addition to the session usage information, the application usage information, the interaction information, and/or other information.

In some implementations, the stated information may include acknowledgements of particular ones of the conditions, illnesses, disorders, and/or other information. The current mental state and the current emotional state may affect the conditions, illnesses, disorders, severity thereof, and how such present themselves by way of the subject. Based on the acknowledgements, health assessment information determination component 112 may be configured to focus on aspects of the psychological profile, the session usage information, and/or other information that are related to those acknowledgements or the aspects that otherwise indicate the acknowledged conditions, illnesses, disorders.

In some implementations, the current mental state and/or the current emotional state may be quantified or represented via the psychological profile. Thus, the determined changes to the psychological profile of the subject (i.e., the psychological parameter values to one or more of the psychological parameters) may be based on the session usage information, the acknowledgements, a past psychological profile (i.e., previously determine psychological parameter values), and/or the other information described herein. Such changes may be presented and/or updated upon the determination.

In some implementations, the determined health assessment information may facilitate determination of healthcare. The healthcare may be determined by health assessment information determination component 112. In some implementations, the healthcare may be determined by the caregiver(s) based on the health assessment information. The healthcare may treat the disorders, illnesses, and/or conditions of the subject, and may improve the current mental state and/or the current emotional state. By way of non-limiting example, the healthcare may include treatment, compliance of the treatment by the subject, condition verification for the subject, prediction of potential outcomes, preemptive actions for the subject and/or caregivers of the subject, and/or other healthcare.

The determined health assessment information may facilitate in adjustment or assignment of the treatment that the subject may comply with based on the determined health assessment information. That is, in some implementations, the treatment of the subject may be adjusted based on the determined health assessment information so that the subject better complies. In some implementations, new treatment may be determined and assigned based on the determined health assessment information such that the subject may comply. By way of non-limiting example, the treatment may include frequent physical activity, medication, therapy, focusing activities, meditation, utilizing particular devices (e.g., sound machine, blue lights, red lights), and/or other treatments.

The determined health assessment information may verify that the identified disorders, illnesses, and/or conditions of the subject are still present, verify the severity of such, and/or may indicate a change in the severity. The potential outcomes may be in response to the determined health assessment information. For example, the determined health assessment information may determine that the subject has a first condition (e.g., an eating disorder). The first condition may cause intestinal damage and further worsen their social connectedness and thus social well-being upon a lack of treatment or lack of compliance with the treatment.

The preemptive actions may address the probable disorders, illnesses, and/or conditions of the subject determined. As such, the probable disorders, illnesses, and/or conditions of the subject may not arise, may arise more slowly, may be controlled during a minor severity. By way of non-limiting example, the preemptive actions may include one or more of the treatments. The preemptive actions may be less severe than an assignment of the treatment. Meaning, for example, the preemptive actions determined may be a lose dose medication to prevent occurrence or worsen of a condition rather than a higher dose medication typically needed for treatment of the condition.

In some implementations, caregiver interface component 118 may be configured to effectuate presentation of a caregiver interface. The caregiver interface may present the health assessment information and/or notifications related to the health assessment information via client computing platforms 104 associated with caregivers for the subject. In some implementations, the caregiver interface may include the content requiring the responses from the subjects so that the caregivers may select which of the content requiring the response to present to the subject during the session. In some implementations, via the caregiver interface, the caregiver may input other information that may facilitate with the health assessment information determination, the healthcare, and/or other information related to the subject.

In some implementations, caregiver interface component 118 may be configured to receive assessment schedule information via the caregiver interface. The assessment schedule information may include one or more of a date, a time, and/or frequency of the determination of the health assessment information for the subject or a group of subjects. In some implementations, the assessment schedule information may specify that the health assessment information may be determined only upon a caregiver authorization. The caregiver authorization may require credentials or identifying information from the caregiver to authorize the determination of the health assessment information. For example, during an in-office visit, the caregiver may provide the caregiver authorization for the session to be began by the subject during the in-office visit.

The health assessment information may be determined in accordance with a frequency. For example, the assessment schedule information may specify that the health assessment information may be determined every Monday, and thus the subject may initiation the session. Based on the assessment schedule information that subject may receive a reminder via their client computing platform 104 to begin the session. The reminder may be persistent or may be reoccurring. The reminder may persistently flash, provide a sound, and/or provide other persistent indications until the subject has initiated the session. The reminder may reoccur after a time amount has lapsed until the subject has initiated the session.

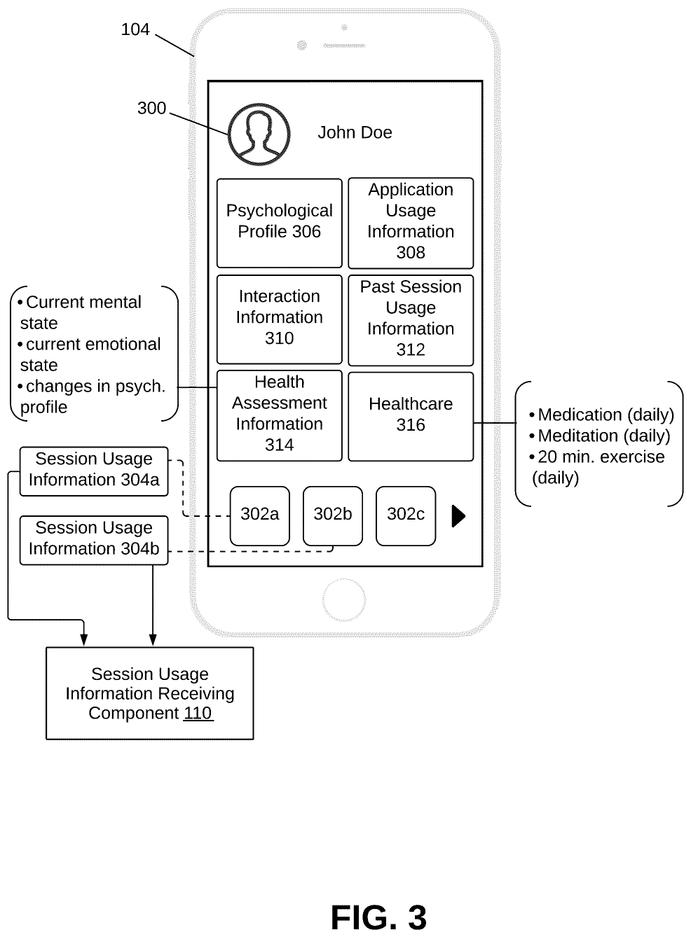

FIG. 3 illustrates an example implementation. A subject 300 may be associated with or interact with client computing platform 104 (e.g., a smart phone). Client computing platform 104 may provide access to online application 302a, 302b, 302c, and/or other online applications. In some implementations, subject 300 may initiate a session via online application 302c. Responsively, session usage information 304a and 304b may be transmitted from online application 302a, 302b, and/or other online applications, respectively, in accordance with a duration of the session upon subject 300 selecting and interacting with such. In some implementations, subject 300 may initiate the session within online application 302a or 302b. The session initiated with online application 302a or 302b may be exclusive to that online application. Responsively, session usage information 304a and 304b may be transmitted from online application 302a or 302b. Session usage information 304a and 304b may be transmitted to session usage information receiving component 110 of system 100 illustrated in FIG. 1. Upon receipt by system 100, session usage information 304a and 304b in addition to one or more of psychological profile 306 of subject 300, application usage information 308 of subject 300, interaction information 310 of subject 300, and/or past session usage information 312 of subject 300 may be utilized to determine health assessment information 314.

Health assessment information 314 may include current mental state, current emotional state, changes in psychological profile 306, and/or other information related to subject 300. Based on health assessment information 314, healthcare 316 may be determined and/or adjusted for subject 300.

Referring to FIG. 1, in some implementations, server(s) 102, client computing platform(s) 104, and/or external resources 122 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which server(s) 102, client computing platform(s) 104, and/or external resources 122 may be operatively linked via some other communication media.

A given client computing platform 104 may include one or more processors configured to execute computer program components. The computer program components may be configured to enable an expert or user associated with the given client computing platform 104 to interface with system 100 and/or external resources 122, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 122 may include sources of information outside of system 100, external entities participating with system 100, and/or other resources. In some implementations, some or all of the functionality attributed herein to external resources 122 may be provided by resources included in system 100.

Server(s) 102 may include electronic storage 124, one or more processors 126, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 124 may comprise non-transitory storage media that electronically stores information. The electronic storage media of electronic storage 124 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 124 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 124 may include one or more virtual storage resources (e.g., cloud storage, a virtual private network, and/or other virtual storage resources). Electronic storage 124 may store software algorithms, information determined by processor(s) 126, information received from server(s) 102, information received from client computing platform(s) 104, and/or other information that enables server(s) 102 to function as described herein.

Processor(s) 126 may be configured to provide information processing capabilities in server(s) 102. As such, processor(s) 126 may include one or more of a digital processor, an analog processor, a digital circuit designed to process information, an analog circuit designed to process information, a state machine, and/or other mechanisms for electronically processing information. Although processor(s) 126 is shown in FIG. 1 as a single entity, this is for illustrative purposes only. In some implementations, processor(s) 126 may include a plurality of processing units. These processing units may be physically located within the same device, or processor(s) 126 may represent processing functionality of a plurality of devices operating in coordination. Processor(s) 126 may be configured to execute components 110, 112, 116, and/or 118, and/or other components. Processor(s) 126 may be configured to execute components 110, 112, 116, and/or 118, and/or other components by software; hardware; firmware; some combination of software, hardware, and/or firmware; and/or other mechanisms for configuring processing capabilities on processor(s) 126. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 110, 112, 116, and/or 118 are illustrated in FIG. 1 as being implemented within a single processing unit, in implementations in which processor(s) 126 includes multiple processing units, one or more of components 110, 112, 116, and/or 118 may be implemented remotely from the other components. The description of the functionality provided by the different components 110, 112, 116, and/or 118 described below is for illustrative purposes, and is not intended to be limiting, as any of components 110, 112, 116, and/or 118 may provide more or less functionality than is described. For example, one or more of components 110, 112, 116, and/or 118 may be eliminated, and some or all of its functionality may be provided by other ones of components 110, 112, 116, and/or 118. As another example, processor(s) 126 may be configured to execute one or more additional components that may perform some or all of the functionality attributed below to one of components 110, 112, 116, and/or 118.

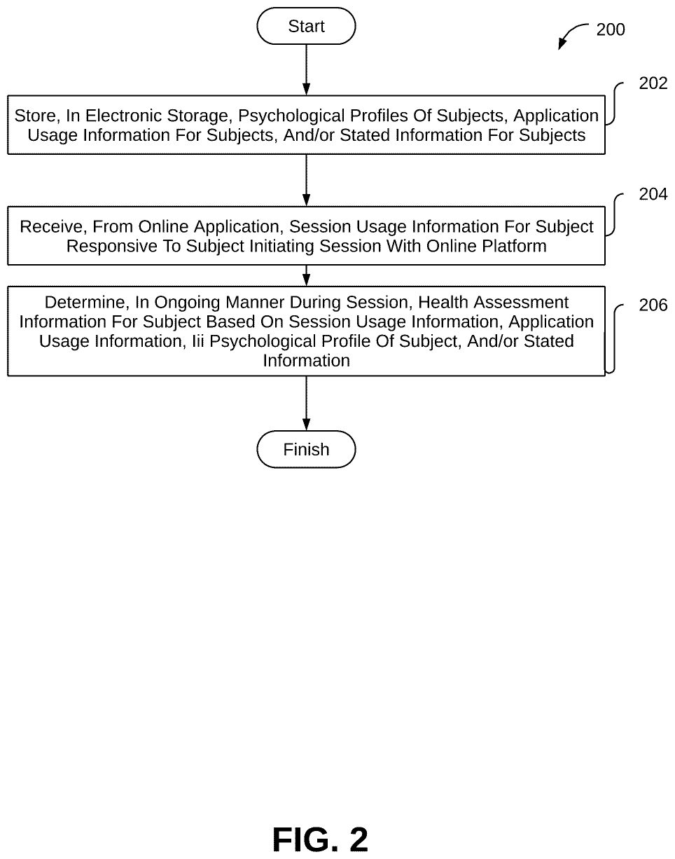

FIG. 2 illustrates a method 200 to assess health information for a subject based on subject-initiated sessions with online applications, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include storing, in electronic storage, psychological profiles of subjects, application usage information for the subjects, and/or stated information for the subjects. The psychological profiles may be determined based on the application usage information and/or the stated information previously obtained for the subjects. Operation 202 may be performed by electronic storage 124, in accordance with one or more implementations.

An operation 204 may include receiving, from an online application, session usage information for a subject responsive to the subject initiating a session with the online platform. The session usage information may characterize current use of the online application by the subject. Operation 204 may be performed by one or more hardware processors configured by machine-readable instructions including a component that is the same as or similar to session usage information receiving component 110, in accordance with one or more implementations.

An operation 206 may include determining, in an ongoing manner during the session, health assessment information for the subject based on the session usage information, the application usage information, a psychological profile of the subject, and/or the stated information. Operation 206 may be performed by one or more hardware processors configured by machine-readable instructions including a component that is the same as or similar to health assessment information determination component 112, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A system configured to assess health information for a subject based on subject-initiated sessions with online applications, the system comprising:
electronic storage configured to store psychological profiles of subjects, application usage information for the subjects, and/or stated information for the subjects, wherein the psychological profiles are determined based on the application usage information and/or the stated information previously obtained for the subjects; and
one or more processors configured by machine-readable instructions to:
receive, from an online application, session usage information for a subject responsive to the subject initiating a session with the online application, the session usage information characterizing current use of the online application by the subject; and
determine, in an ongoing manner during the session, health assessment information for the subject based on i) the session usage information, ii) the application usage information, iii) a psychological profile of the subject, and/or iv) the stated information.

2. The system of claim 1, wherein the health assessment information includes a current mental state and/or a current emotional state, and/or changes to psychological parameter values to psychological parameters that comprise the psychological profile.

3. The system of claim 1, wherein the session usage information includes game mechanics within the online application, one or more games chosen to be played, social interactions, read literature, and/or unread literature.

4. The system of claim 3, wherein the game mechanics include movements, alternating turns, playing cards, dice, role-playing, and/or action points.

5. The system of claim 1, wherein the determined health assessment information facilitates determination of healthcare, wherein the healthcare includes compliance of treatment by the subject, condition verification for the subject, prediction of potential outcomes, and/or preemptive actions for the subject and/or caregivers of the subject.

6. The system of claim 1, wherein the one or more processors are further configured by the machine-readable instructions to:
receive an indication that the session has been initiated by the subject; and
effectuate, via the online application, content that require one or more responses from the subject, wherein determining the health assessment information is based on the one or more responses.

7. The system of claim 1, wherein the one or more processors are further configured by the machine-readable instructions to:
effectuate presentation of a caregiver interface, the caregiver interface presenting the health assessment information and/or notifications related to the health assessment information via client computing platforms associated with caregivers for the subject.

8. The system of claim 7, wherein the one or more processors are further configured by the machine-readable instructions to receive assessment schedule information via the caregiver interface, the assessment schedule information including one or more of a date, a time, and/or frequency of the determination of the health assessment information for the subject or a group of subjects.

9. The system of claim 6, wherein the content includes timed challenges, written response questions, riddles, visual games, auditory games, and/or memory games, wherein the content is generated and/or selected by one or more caregivers of the subject or is an item within the online application.

10. A method to assess health information for a subject based on subject-initiated sessions with online applications, the method comprising:
storing, in electronic storage, psychological profiles of subjects, application usage information for the subjects, and/or stated information for the subjects, wherein the psychological profiles are determined based on the application usage information and/or the stated information previously obtained for the subjects;
receiving, from an online application, session usage information for a subject responsive to the subject initiating a session with the online application, the session usage information characterizing current use of the online application by the subject; and
determining, in an ongoing manner during the session, health assessment information for the subject based on i) the session usage information, ii) the application usage information, iii) a psychological profile of the subject, and/or iv) the stated information.

11. The method of claim 10, wherein the health assessment information includes a current mental state and/or a current emotional state, and/or changes to psychological parameter values to psychological parameters that comprise the psychological profile.

12. The method of claim 10, wherein the session usage information includes game mechanics within the online application, one or more games chosen to be played, social interactions, read literature, and/or unread literature.

13. The method of claim 12, wherein the game mechanics include movements, alternating turns, playing cards, dice, role-playing, and/or action points.

14. The method of claim 10, wherein the determined health assessment information facilitates determination of healthcare, wherein the healthcare includes compliance of treatment by the subject, condition verification for the subject, prediction of potential outcomes, and/or preemptive actions for the subject and/or caregivers of the subject.

15. The method of claim 10, further comprising:
receiving an indication that the session has been initiated by the subject; and
effectuating, via the online application, content that require one or more responses from the subject, wherein determining the health assessment information is based on the one or more responses.

16. The method of claim 10, further comprising:
effectuating presentation of a caregiver interface, the caregiver interface presenting the health assessment information and/or notifications related to the health assessment information via client computing platforms associated with caregivers for the subject.

17. The method of claim 16, further comprising receiving assessment schedule information via the caregiver interface, the assessment schedule information including one or more of a date, a time, and/or frequency of the determination of the health assessment information for the subject or a group of subjects.

18. The method of claim 15, wherein the content includes timed challenges, written response questions, riddles, visual games, auditory games, and/or memory games, wherein the content is generated and/or selected by one or more caregivers of the subject or is an item within the online application.
